(19) United States
(12) Patent Application Publication
(10) Pub. No.: US 2019/0154684 A1
(43) Pub. Date: May 23, 2019

(54) APPARATUS FOR VISUAL DETECTION OF ANALYTES IN A LIQUID SAMPLE

(71) Applicant: Protzek Gesellschaft fuer Biomedizinische Technik GmbH, Pratteln (CH)

(72) Inventor: Christoph PROTZEK, Loerrach (DE)

(73) Assignee: Protzek Gesellschaft fuer Biomedizinische Technik GmbH, Pratteln (CH)

(21) Appl. No.: 16/320,709

(22) PCT Filed: Aug. 23, 2017
(86) PCT No.: PCT/EP2017/071186

§ 371 Date: Jan. 25, 2019

(30) Foreign Application Priority Data
Aug. 24, 2016 (EP) 16185557.2

(51) Int. Cl. G01N 33/558; G01N 33/487

(52) U.S. Cl. CPC G01N 33/558; G01N 33/4875

(57) ABSTRACT

An apparatus for visual detection of analytes in an analytical or liquid sample originating from a human or animal organism includes a carrier plate having at least one test strip receiver in which a test strip provided with a sample receiving section and a test section is arranged, the at least one test strip receiver being sealed at least sectionwise via a covering layer non-detachably joined to the carrier plate, the covering layer having an examination window in the region of the test section of the test strip in the at least one test strip receiver.

The invention relates to an apparatus for visual detection of analytes in the form of an analysis sample or liquid sample originating from a human or animal organism, in accordance with the preamble of claim 1.

Apparatuses for detection of analytes in a liquid sample, for example of drugs, medications or antibodies as indicators of specific illnesses in a bodily fluid or determinations in the sense of concentration data are known in different embodiments. Particular interest exists in those devices that can also be used outside of a laboratory. These must be portable and easy to operate, and also must yield a reliable result within a short period of time. In particular for conducting tests for drug use or medication abuse or the like by the police, an apparatus is required that allows such a test even outdoors, under different and varying light conditions, weather conditions, and temperatures. In DE 20 2014 002 369 U1, a device is proposed for this purpose, into which a test cassette can be introduced, and which is provided with a light source by means of which the lower wall of the test cassette, which consists of a translucent plastic, as well as the test region of the test strip disposed in the test cassette irradiated and the test result lights up. The test cassette is designed for carrying out a lateral flow immunoassay, aptamer-based assay, or enzyme immunoassay (EIA), and consists of a lower lid and an upper lid. Enclosed by these two lids, the test cassette contains a test strip, which is held in position within a holder. In the upper lid, there is a fill-in opening above the sample holder of the test strip, through which the liquid sample to be examined can be introduced. There is a monitoring window above the center test region of the test strip, through which the test result can be read. Such cassettes have proven themselves in mobile use. However, it is a disadvantage of the previously known cassettes that its production is complicated and that they require a lot of space.

This is where the invention wishes to provide a remedy. The invention is based on the task of making available an apparatus for visual and measurement-technology detection of analytes in a liquid sample, which has a simple structure, is suitable for mobile use, and only requires a small amount of space. According to the invention, this task is accomplished by means of an apparatus having the characteristics of claim 1.

With the invention, an apparatus for visual and measurement-technology detection of analytes in a liquid sample is made available, which has a simple structure, is suitable for mobile use, and only requires a small amount of space. A very space-saving, compact construction is made possible by means of the configuration of the apparatus by means of a carrier plate that is provided with a test strip holder, in which plate a test strip having a sample receiving section and a test section is disposed, which at least one test strip holder is closed off by way of a cover layer connected with the carrier plate in non-releasable manner, at least in certain regions. Preferably, the cover layer has a sample application window, which allows sample application to the sample receiving section of at least one test strip, disposed at a distance from the monitoring window.

In a further development of the invention, at least two test strip holders disposed parallel to one another, each holding one test strip, are provided, which holders open into a common reservoir at one end, in which reservoir the sample receiving sections of the test strips are disposed, and above which reservoir the sample application window of the cover layer is positioned. As a result, simultaneous sample application to all the sample receiving sections of the test strips that open into the reservoir is made possible. Furthermore, sufficient feed of liquid sample is guaranteed by way of the configuration of the reservoir, as is homogeneous distribution of the liquid on the sample receiving section of the test strips, which section is preferably provided with a separation film (sample nonwoven).

In an embodiment of the invention, a sample receiving nonwoven is disposed in the region of the sample application window, between cover layer and test strip. As a result, uniform impact of the liquid sample on the test strip is brought about.

In a further embodiment of the invention, the carrier plate has a section that can pivot along a bending fold, which section is not provided with the cover layer in the region of the test strips, and which section is disposed in such a manner that the sample receiving section of the test strips projects out of the carrier plate, at least in certain regions, after the section has been bent away. As a result, carrying out a dip test is made possible.

In a further development of the invention, a lid is disposed on the carrier plate, which lid is connected with the plate so as to pivot. As a result, sufficient protection of the regions of the test strips not provided with the cover layer is achieved. Preferably, the lid is formed onto the carrier plate in one piece with it.

In a further embodiment of the invention, the carrier plate is produced from cardboard, wherein existing test strip holders and/or reservoirs are formed by means of embossing. As a result, environmentally friendly disposal of the apparatus is achieved. For this purpose, the lid is also preferably made of cardboard. In an alternative embodiment of the invention, existing test strip holders and/or reservoirs can also be formed by means of recesses of an intermediate layer disposed between cover film and carrier plate.

In a further development of the invention, the cover layer is formed by a film that is adhesively attached to the carrier plate and the test strips held by the carrier plate, preferably in essentially full-area manner. As a result, protection of the test strips against contamination is achieved. In this regard, it is advantageous if the film is produced from degradable material. It is advantageous if the at least one monitoring window is formed by a transparent region of the film, in each instance.

In a further embodiment of the invention, two carrier plates connected with one another so as to pivot are provided, which are provided with test strip holders on half of their surface, in each instance, in such a manner that in the folded-in state, the half of a carrier plate provided with test strip holders lies against the half of the other carrier plate that lies against it and does not have any test strip holders. As a result, placement of test strips on both sides of the apparatus is achieved, and thereby the possible number of test strips is increased. In this regard, a thin construction is achieved at the same time.

Other further developments and embodiments of the invention are indicated in the remaining dependent claims. Exemplary embodiments of the invention are shown in the drawings and will be described in detail below. The drawings show:

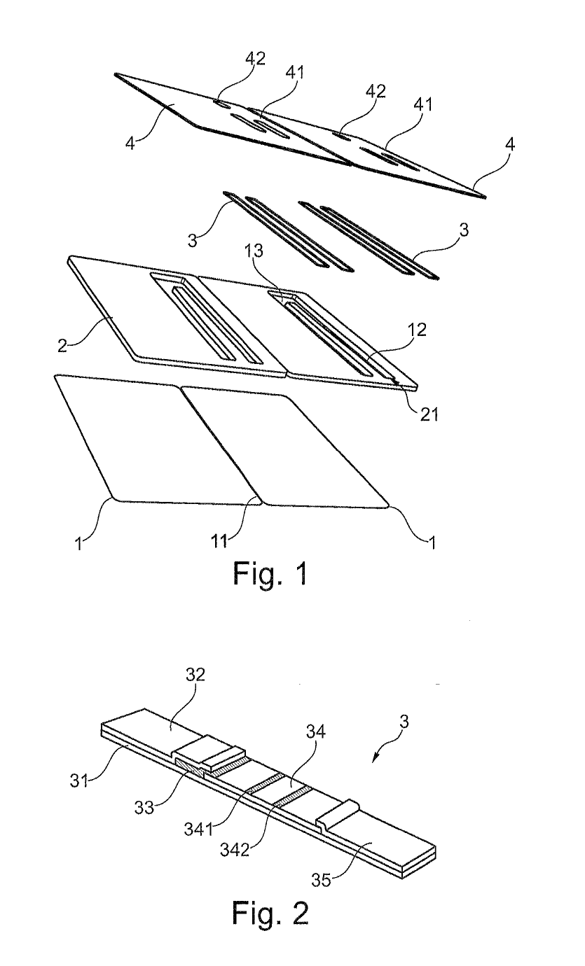

FIG. 1 the schematic representation of an apparatus for visual detection of analytes in a liquid sample, in an exploded representation;

FIG. 2 the schematic representation of a test strip of the apparatus from FIG. 1;

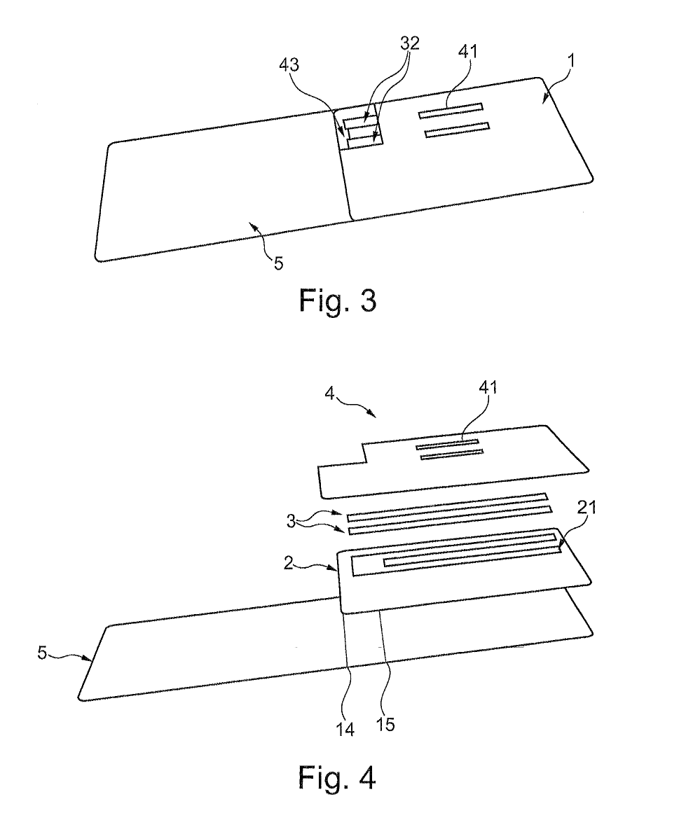

FIG. 3 the schematic representation of an apparatus for visual detection of analytes in a liquid sample, in a further embodiment;

FIG. 4 the representation of the apparatus from FIG. 1 in an exploded representation;

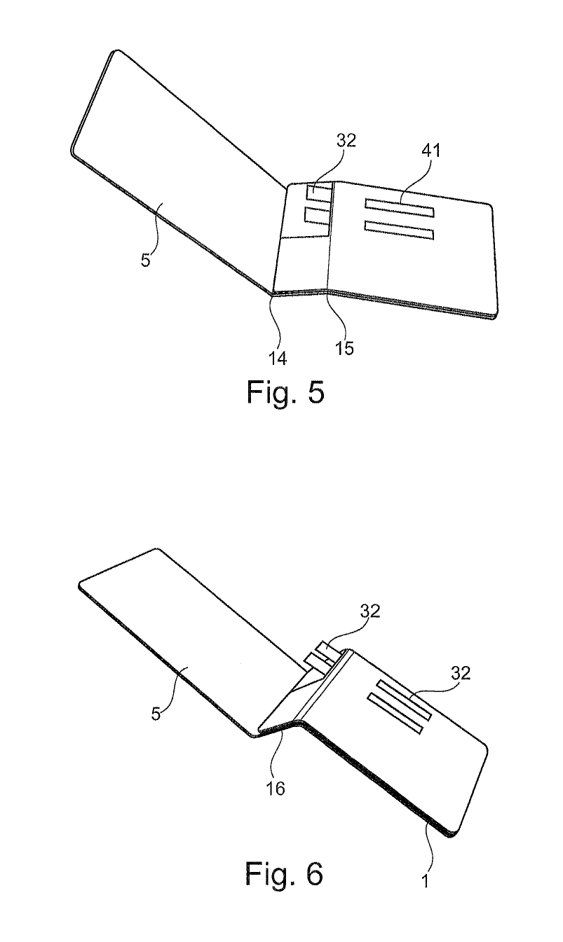

FIG. 5 the schematic representation of the apparatus from FIG. 3 in the bent-away state, and

FIG. 6 the apparatus from FIG. 3 with the sample receiving sections of the test strips exposed.

The apparatus for visual detection of analytes in a liquid sample, selected as an exemplary embodiment, essentially consists of two carrier plates 1 that are connected with one another, so as to pivot, along their longitudinal side, onto which plates an intermediate layer 2 is applied, in each instance, holds the test strips 3, and which is provided with a cover layer 4.

In the exemplary embodiment, the carrier plates 1 are produced from cardboard, and formed from a single cardboard cutout provided with a bending fold 11 in the center.

The intermediate layer 2 is produced from corrugated cardboard in the exemplary embodiment, and coated with a transparent, liquid-sealing film, so as to prevent penetration of sample liquid into the cardboard. Alternatively, the intermediate layer 2 can also be provided with a water-tight, preferably environmentally safe varnish. A recess 21 is introduced into the intermediate layer 2, by means of which recess two test strip holders 12 are formed, in each instance, which holders open into a reservoir 13. The test strip holders 12 hold test strips 3.

The test strip 3 is shown in stepped section in FIG. 2. The test strip 3 has a carrier film 31, on which a sample receiving section 32 is disposed at one end. An antibody coating 33, which contains antibodies that are conjugated with gold particles and match an analyte to be determined or an antigen of this analyte, and can enter into an immunological reaction is situated on the carrier film 31 directly adjacent and partially below the sample receiving section 32. The antibody coating section 33 is followed by a test section 34, which is configured in the form of a membrane, on which a test line 341 and a monitoring line 342 are situated, at a distance from one another, which lines are configured by means of an antigen contained in a reagent for detection of a specific analyte, and are invisible before a sample is applied. The sample receiving section 32 of the test strip 3 is situated below the sample application window 42, and the test section 34 is situated below the monitoring window 41 of the cover layer 4 applied to the intermediate layer 2.

When carrying out a lateral flow assay, the test line 341 shows the result of the test; the monitoring line 342 indicated whether a sufficient sample amount was applied, in other words whether the test is valid and it was carried out properly. An absorber section 35 is disposed on the test strip 3, on its end that lies opposite the sample receiving section 32.

In the exemplary embodiment, a cover layer 4 is composed of a degradable, colored film, which is provided with transparently configured, clear, low-reflection monitoring windows 41. A sample application window 42, which is formed by a recess introduced into the cover layer 4, is disposed at a distance from the monitoring windows 41. The sample application window 42 is disposed in such a manner that it is positioned above the sample receiving sections 32 of the two test strips 3. In the exemplary embodiment, a sample receiving nonwoven—not shown—is disposed between the test strips 3 and the sample application 42 of the cover layer 4, which nonwoven covers the sample receiving sections 32 of the two test strips 3.

Before a test is carried out, no lines 341, 342 can be seen. While a test is carried out, a sample volume of a sample liquid is applied to the sample receiving section 32 of each test strip 3 through a sample application window 42 of the cover layer 4. The sample liquid flows in the direction of the test section 34 by means of capillary action. In this regard, it first comes into contact with the antibody coating section 33, in which antibodies, conjugated with gold particles, to the antigen of an analyte to be determined are situated in a standardized (metered) amount. The antibodies have different binding activity for different analytes (drugs). The antibodies, conjugated with gold particles, are entrained by the sample liquid that flows through the test section 34. If the sample liquid does not contain the analyte to be determined, then as it flows through the test section 34, the antigens of the analyte that are situated there, in the region of the test line 341, are bound by the antibodies entrained by the sample liquid, and a visible test line 341 forms, which contains a red or brown color due to the gold particles conjugated with the antibodies. If the sample liquid contains the analyte to be determined, then its antigens are already bound by the antibodies in the region of the antibody coating section 33 of the test strip 3, and accumulation can no longer occur in the test section 34, and therefore no visible test line 341 forms. The monitoring line 342 indicates whether the test is valid. In FIG. 2, the test line 341 is formed in color, and therefore the test is negative with regard to the corresponding analyte.

The carrier plates 1, which are connected with one another so as to pivot, by way of the bending fold 11, are pivoted in such a manner that their rear sides, which lie opposite the test strips 3, lie against one another. As a result, an apparatus having approximately the size of a credit card is achieved, which holds four test strips 3. When using usual test strips, which allow the test with regard to three analytes, it is therefore made possible to detect twelve analytes using the apparatus according to the invention, which has the size of a credit card in the exemplary embodiment.

In the exemplary embodiment according to FIG. 3, a carrier plate 1 is provided, which is provided with an intermediate layer 2 in the manner described above, and the test strip holders 12 of this plate hold test strips 3, wherein the intermediate layer 2 is provided with a cover layer 4. In place of a sample application window, a corner region is removed from the cover layer 4, facing the test strip 3, so that the sample application sections 32 of the test strip 3 are essentially not covered by the cover layer 4.

A lid 5 is connected to the carrier plate 1 with the carrier plate 1 by way of a first bending fold 14, so as to pivot, on its transverse side that faces the sample receiving sections 32 of the test strips 3. In the exemplary embodiment, the carrier plate 1 and the lid 5 are formed from a cardboard cutout provided with the first bending fold 14. A second bending fold 15 is introduced into the carrier plate 1, with the intermediate layer 2 provided on it, at a distance from the first bending fold 14, at the level of the recess 43 of the cover layer 4. The front section 16 of the carrier plate 1, which is delimited by the second bending fold 15, can be pivoted by way of this fold, and thereby the sample receiving sections 32 of the two test strips 3 are exposed. To carry out a dip test, the front section 16, together with the lid 5, can be laid against the rear side of the carrier plate 1 in this way. Different bending positions are shown in FIGS. 5 and 6.

In place of the intermediate layer 2 provided with recesses, the carrier plate 1 can also be provided with embossed regions, and thereby the test strip holders 12 and the reservoir 13 are configured in the form of depressions in the carrier plate 1. As a result, the production effort can be further minimized.

The apparatus according to the invention is characterized in that it allows testing of a great number of analytes while having a compact configuration. Furthermore, environmentally friendly disposal of the apparatus, intended for single use, is made possible by means of production of the apparatus essentially from cardboard materials. Furthermore, an apparatus configured according to the second exemplary embodiment simultaneously also allows use for a dip test.

1. An apparatus for visual detection of analytes in an analysis sample or liquid sample originating from a human or animal organism, comprising a carrier plate (1) having at least one test strip holder (12), in which plate a test strip (3) having a sample receiving section (32) and a test section (34) is disposed, which at least one test strip holder (12) is closed off by way of a cover layer (4) connected with the carrier plate (1) in non-releasable manner, at least in certain regions, which cover layer (4) has a monitoring window (41) in the region of the test section (34) of the test strip (3) of the at least one test strip holder (12).

2. The apparatus according to claim 1, wherein the cover layer (4) has a sample application window (42), which allows sample application to the sample receiving section (32) of at least one test strip (3), disposed at a distance from the monitoring window (41).

3. The apparatus according to claim 2, wherein at least two test strip holders (12) disposed parallel to one another, each holding one test strip (3), are provided, which holders open into a common reservoir (13) at one end, in which reservoir the sample receiving sections (32) of the test strips (3) are disposed, and above which reservoir the sample application window (42) of the cover layer (4) is positioned.

4. The apparatus according to claim 2, wherein a sample receiving nonwoven is disposed in the region of the sample application window (42), between cover layer (4) and test strip (3).

5. An apparatus according to claim 1, wherein the carrier plate (1) has a section (16) that can pivot along a bending fold (14), which section is not provided with the cover layer in the region of the test strips (3), and which section is disposed in such a manner that the sample receiving section (32) of the test strips (3) projects out of the carrier plate (1), at least in certain regions, after the section (16) has been bent away.

6. The apparatus according to claim 5, wherein a lid (5) is disposed on the carrier plate (1), which lid is connected with the plate so as to pivot.

7. The apparatus according to claim 6, wherein the lid (5) is formed from cardboard.

8. The apparatus according to claim 6, wherein the lid (5) is formed onto the carrier plate (1) in one piece.

9. The apparatus according to claim 1, wherein the carrier plate (1) is produced from cardboard, wherein existing test strip holders (12) and/or reservoirs (13) are formed by means of embossing.

10. The apparatus according to claim 1, wherein existing test strip holders (12) and/or reservoirs (13) are formed by means of recesses (21) of an intermediate layer (2) disposed between cover film (4) and carrier plate (1).

11. The apparatus according to claim 1, wherein the cover layer (4) is formed by a film that is adhesively attached to the carrier plate (1) and the test strips (3) held by the carrier plate (1), preferably in essentially full-area manner.

12. The apparatus according to claim 1, wherein the at least one monitoring window (41) is formed by at least one transparent region of the cover layer (4), in each instance.

13. The apparatus according to claim 1, wherein the two carrier plates (1) connected with one another so as to pivot are provided, which are provided with test strip holders (12) on half of their surface, in each instance, in such a manner that in the folded-in state, the half of a carrier plate (1) provided with test strip holders (12) lies against the half of the other carrier plate (1) that lies against it and does not have any test strip holders (12).
